(19) United States
(12) Patent Application Publication
(10) Pub. No.: US 2013/0225961 A1
(43) Pub. Date: Aug. 29, 2013

(54) DIALYSIS PROBE

(71) Applicants: Hirohito NISHINO, Kyoto (JP); Kazuko IKIMURA, Kyoto (JP); Shuko TAKEDA, Osaka (JP); Naoyuki SATO, Osaka (JP); Ryuichi MORISHITA, Osaka (JP)

(72) Inventors: Hirohito NISHINO, Kyoto (JP); Kazuko IKIMURA, Kyoto (JP); Shuko TAKEDA, Osaka (JP); Naoyuki SATO, Osaka (JP); Ryuichi MORISHITA, Osaka (JP)

(73) Assignees: OSAKA UNIVERSITY, Osaka (JP); EICOM CORPORATION, Kyoto (JP)

(21) Appl. No.: 13/792,985

(22) Filed: Mar. 11, 2013

Related U.S. Application Data
(62) Division of application No. 13/089,536, filed on Apr. 19, 2011.

(30) Foreign Application Priority Data
Apr. 26, 2010 (JP) 2010100530

(51) Int. Cl. A61B 5/145

(52) U.S. Cl. CPC A61B 5/14528
USPC 600/366

(57) ABSTRACT

A dialysis probe includes a tubular dialysis membrane sealed at its tip, a support tube coupled at a tip to a rear end of the dialysis membrane, a cap portion for securing a rear end of the support tube, an inlet conduit extending through the cap portion toward the tip of the dialysis membrane within a space to guide a perfusate into the space, an outlet conduit extending through the cap portion toward the tip of the dialysis membrane within the space to guide the perfusate in the space outside the space, and at least one air-exposure through-hole provided in the cap portion for maintaining the space at atmospheric pressure. The inlet conduit has a longer protruding length from the rear end of the support tube when compared to the outlet conduit.

### BACKGROUND OF THE INVENTION

1. Field of the Invention

The present invention relates to a dialysis probe to be embedded in a body tissue when used for collecting various biological materials included in the body tissue by microdialysis.

2. Description of Background

Microdialysis is a known method for analyzing various biological materials included in body tissues such as brains, muscles, skin, kidneys, and blood vessels. Microdialysis is a method in which an artificial tissue fluid is forced to flow as a perfusate inside a dialysis membrane embedded in a body tissue, thereby dialytically collecting biological materials included in the body tissue into the perfusate in a successive manner by simple diffusion. By analyzing the compositions of collected biological materials, it becomes possible to estimate activities of body tissues, thereby understanding various physiological activities of living organisms.

This method uses a dialysis probe with a tubular dialysis membrane at its tip, but when collecting high-molecular-weight biological materials using a dialysis membrane with a molecular-weight cutoff (a cutoff value, also referred to as MWCO) of 50,000 Da or more, it is often the case that a push-pull dialysis probe is particularly used to prevent a perfusate from leaking through the dialysis membrane into a body tissue.

For example, Patent Document 1 describes a conventionally known push-pull dialysis probe. This dialysis probe includes an inlet conduit and an outlet conduit, the inlet conduit guiding a perfusate fed from an infusion pump into a closed space formed by a tubular dialysis membrane and a support tube, the outlet conduit guiding the perfusate in the closed space to a suction pump located outside the closed space.

[Patent Document 1] Japanese Patent No. 2866301

### SUMMARY OF THE INVENTION

Incidentally, in the case of analyzing biological materials using microdialysis, it is important to be always able to continuously collect a constant amount of biological material during an experiment of several hours unless the amount (concentration) of biological material included in a body tissue changes. Therefore, in the case where a push-pull dialysis probe is used, it is necessary to completely equalize the flow rate of an inflow perfusate entering through the inlet conduit with the flow rate of an outflow perfusate (including biological materials) exiting through the outlet conduit. If the balance between these flow rates is lost, the closed space is pressurized/depressurized, so that the amount (concentration) of biological material collected temporarily increases/decreases and the perfusate leaks into the body tissue to adversely affect the body tissue, which makes it impossible to stably perform an analysis with high accuracy.

However, in general, the flow rate of a perfusate is as extremely low as several μL/min., and furthermore, pipes for connecting pumps to a dialysis probe are about 0.1 to 0.3 mm in inner diameter and tens of cm or more in length and therefore are very elongated. Accordingly, conventional push-pull dialysis probes have difficulties in completely equalizing the inflow and outflow rates of a perfusate and therefore are prone to variations in the amount (concentration) of biological material collected.

The present invention has been made in view of the circumstances as mentioned above, and an objective thereof is to provide a dialysis probe capable of stably performing an analysis with high accuracy for a long period of time.

To solve the problems mentioned above, a dialysis probe according to a first aspect of the present invention includes (i) a tubular dialysis membrane sealed at its tip; (ii) a support tube coupled at a tip to a rear end of the dialysis membrane; (iii) a cap portion for securing a rear end of the support tube; (iv) an inlet conduit for guiding a perfusate into a space enclosed by the dialysis membrane, the support tube, and the cap portion, the inlet conduit extending through the cap portion toward the tip of the dialysis membrane within the space; (v) an outlet conduit for guiding the perfusate in the space outside the space, the outlet conduit extending through the cap portion toward the tip of the dialysis membrane within the space; and (vi) at least one air-exposure through-hole provided in the cap portion for maintaining the space at atmospheric pressure, the inlet conduit has a longer protruding length from the rear end of the support tube when compared to the outlet conduit, and an outflow rate of the perfusate exiting through the outlet conduit is set to be greater than or equal to an inflow rate of the perfusate entering through the inlet conduit.

In the first aspect, the dialysis membrane and the support tube are joined, for example, by an adhesive layer provided between an outer circumferential surface of the dialysis membrane and an inner circumferential surface of the support tube.

To solve the problems mentioned above, a dialysis probe according to a second aspect of the present invention includes (i) a tubular dialysis membrane open at opposite ends; (ii) an inlet conduit coupled at a tip to one end of the dialysis membrane so as to guide a perfusate into a space within the dialysis membrane; and (iii) an outlet conduit for guiding the perfusate in the space within the dialysis membrane outside the space, the outlet conduit extending from the space in an opposite direction to the inlet conduit, the space is exposed to air, and an outflow rate of the perfusate exiting through the outlet conduit is set to be greater than or equal to an inflow rate of the perfusate entering through the inlet conduit.

The dialysis probe according to the second aspect further includes (iv) an air-exposure tube extending from the space in the same direction as the outlet conduit or (iv′) an extension tube concentrically coupled to the other end of the dialysis membrane and extending in an axial direction of the dialysis membrane, and the outlet conduit extends inside the extension tube.

In the second aspect, the inlet conduit and the dialysis membrane are joined, for example, by an adhesive layer provided between an outer circumferential surface of the inlet conduit and an inner circumferential surface of the dialysis membrane.

In the second aspect, the extension tube and the dialysis membrane are joined, for example, by an adhesive layer provided between an outer circumferential surface of the extension tube and an inner circumferential surface of the dialysis membrane. Also, in the second aspect, the extension tube and the outlet conduit are partially joined, for example, by an adhesive layer provided between a portion of an inner circumferential surface of the extension tube and a portion of an outer circumferential surface of the outlet conduit.

The present invention makes it possible to provide a dialysis probe capable of stably performing an analysis with high accuracy for a long period of time even if the inflow and outflow rates of a perfusate are not completely equalized.

### BRIEF DESCRIPTION OF THE DRAWINGS

FIG. 1 provides cross-sectional views of a dialysis probe according to a first embodiment: (A) an overall cross-sectional view; (B) and (C) partial cross-sectional views each illustrating a variant on a portion for coupling a dialysis membrane and a support tube.

FIG. 2 is a schematic diagram illustrating the dialysis probe according to the first embodiment in use.

FIG. 3 is a diagram describing the operating principle of the dialysis probe according to the first embodiment.

FIG. 4 provides cross-sectional views of a dialysis probe according to a second embodiment: (A) an overall cross-sectional view; (B) to (D) partial cross-sectional views each illustrating a variant on a joint portion of an outlet conduit.

FIG. 5 is a schematic diagram illustrating the dialysis probe according to the second embodiment in use.

### DETAILED DESCRIPTION OF THE INVENTION

Hereinafter, preferred embodiments of a dialysis probe according to the present invention will be described with reference to the accompanying drawings.

### First Embodiment

### Type for Brain Tissue

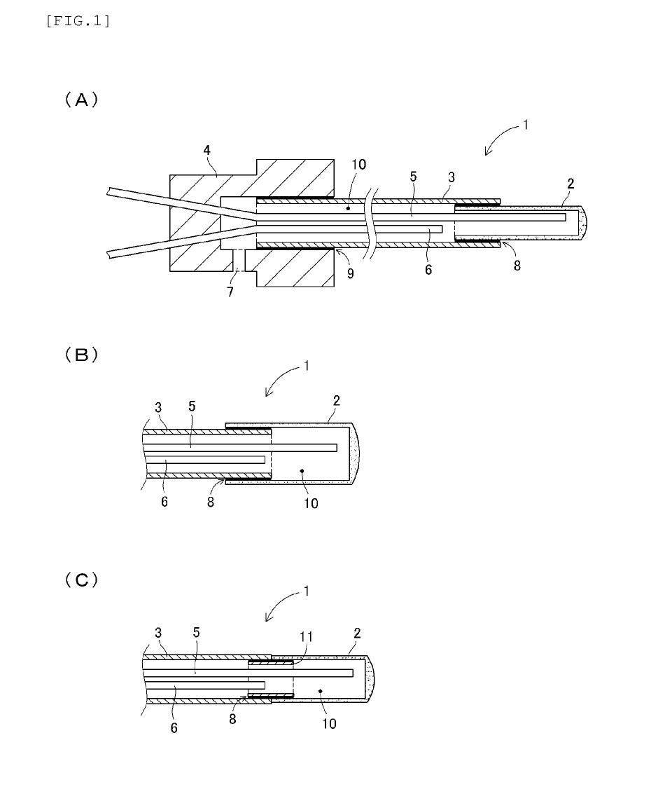

A dialysis probe 1 according to a first embodiment of the present invention is mainly used for collecting and analyzing biological materials in brain tissues, and includes a tubular dialysis membrane 2 sealed at its tip, a support tube 3 coupled at a tip to a rear end of the dialysis membrane 2, a cap portion 4 for securing a rear end of the support tube 3, an inlet conduit 5 extending through the cap portion 4 toward the tip of the dialysis membrane 2 within a space 10 enclosed by the dialysis membrane 2, the support tube 3, and the cap portion 4, an outlet conduit 6 extending through the cap portion 4 toward the tip of the dialysis membrane 2 within the space 10, and an air-exposure through-hole 7 provided in the cap portion 4, as shown in FIG. 1(A). Furthermore, when compared to the outlet conduit 6, the inlet conduit 5 has a longer protruding length from the rear end of the support tube 3.

The dialysis membrane 2 is a hollow fiber dialysis membrane with an outer diameter of hundreds of pm and a thickness of tens of μm. In the present embodiment, a tubular dialysis membrane for use in artificial dialysis is cut to a predetermined length, and then sealed at its tip with proper resin to be used as a dialysis membrane 2. In the present invention, the dialysis membrane 2 is not specifically limited in terms of its material, dimensions and molecular-weight cutoff, and various kinds of dialysis membrane can be used.

The support tube 3 is a tubular member made of a more rigid material (e.g., stainless steel or silica glass) than the dialysis membrane 2. As shown in FIG. 1(A), the inner diameter of the support tube 3 is slightly larger than the outer diameter of the dialysis membrane 2, and the outer circumferential surface of the dialysis membrane 2 and the inner circumferential surface of the support tube 3 are joined by an adhesive layer 8 provided therebetween. The adhesive layer 8 is made of, for example, an epoxy-based adhesive.

There are a variety of conceivable variants on the coupling form of the dialysis membrane 2 and the support tube 3. For example, as shown in FIG. 1(B), the inner diameter of the dialysis membrane 2 may be set to be slightly larger than the outer diameter of the support tube 3, so that the inner circumferential surface of the dialysis membrane 2 and the outer circumferential surface of the support tube 3 can be joined by the adhesive layer 8. Alternatively, as shown in FIG. 1(C), the dialysis membrane 2 and the support tube 3 may be approximately equalized in inner diameter, and the inner circumferential surface of the dialysis membrane 2 and the inner circumferential surface of the support tube 3 may be joined to the outer circumferential surface of a coupling tube 11 by the adhesive layer 8, so that the dialysis membrane 2 and the support tube 3 are coupled via the coupling tube 11.

The cap portion 4 is made of proper resin such as acrylic resin or epoxy resin, and is joined to the rear end-side outer circumferential surface of the support tube 3 by an adhesive layer 9, thereby securing the rear end of the support tube 3. The inlet conduit 5 and the outlet conduit 6 are embedded in the cap portion 4 and thereby secured in their predetermined positions. The adhesive layer 9 is made of, for example, an epoxy-based adhesive.

The inlet conduit 5 is provided so as to penetrate the cap portion 4 and extend within the space 10 in a direction from the rear end of the support tube 3 to the tip of the dialysis membrane 2. The inlet conduit 5 is a thin tube made of, for example, silica glass, and its external portion leading from the cap portion 4 is preferably covered and reinforced by an unillustrated stainless-steel tube. Also, from the viewpoint of dialysis efficiency, the inlet conduit 5 preferably extends as close as possible to the tip of the dialysis membrane 2.

The outlet conduit 6 is provided so as to penetrate the cap portion 4 and extend within the space 10 in a direction from the rear end of the support tube 3 to the rear end of the dialysis membrane 2. Specifically, when compared to the inlet conduit 5, the outlet conduit 6 has a shorter protruding length from the rear end of the support tube 3, as shown in FIG. 1(A). The outlet conduit 6 is a thin tube made of, for example, silica glass, and its external portion leading from the cap portion 4 is preferably covered and reinforced by an unillustrated stainless-steel tube.

The cap portion 4 has provided therein the air-exposure through-hole 7 with a diameter of 0.7 mm, which leads to the space 10. Accordingly, the space 10 is an open space, rather than a closed space, and the pressure within the space 10 is maintained at a level (atmospheric pressure) around the dialysis probe 1. Note that in the present embodiment, one air-exposure through-hole 7 is provided in the side surface of the cap portion 4, but this is simply an example and more than one air-exposure through-hole 7 may be provided in another place. Also, the diameter of the air-exposure through-hole 7 is not limited to 0.7 mm and can be appropriately changed.

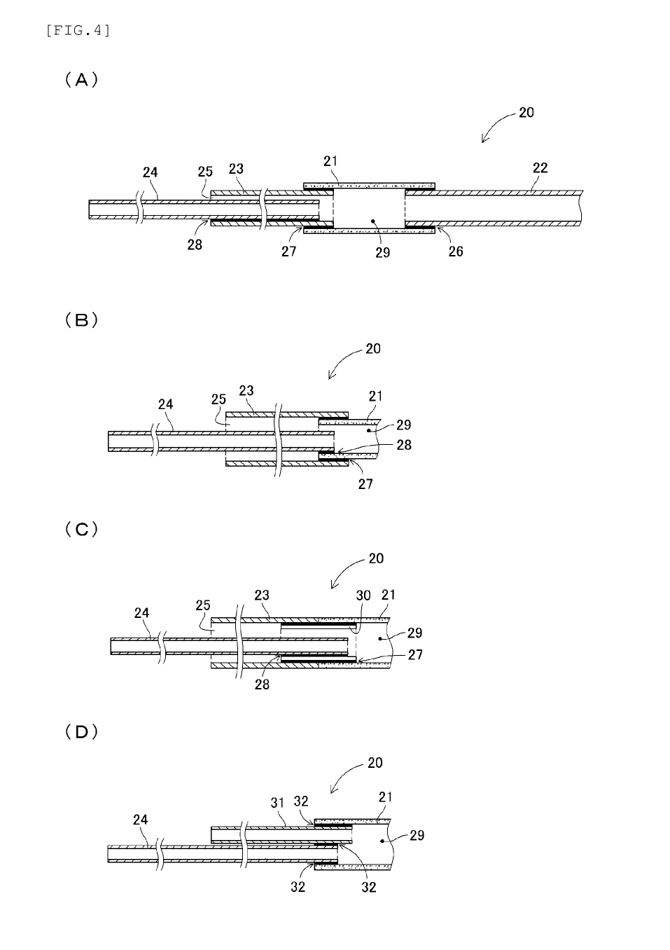

In addition, if the space 10 can be an open space, an air-exposure tube (see FIG. 4(D)) may be embedded and fixed in the cap portion 4, instead of providing the air-exposure through-hole 7 in the cap portion 4.

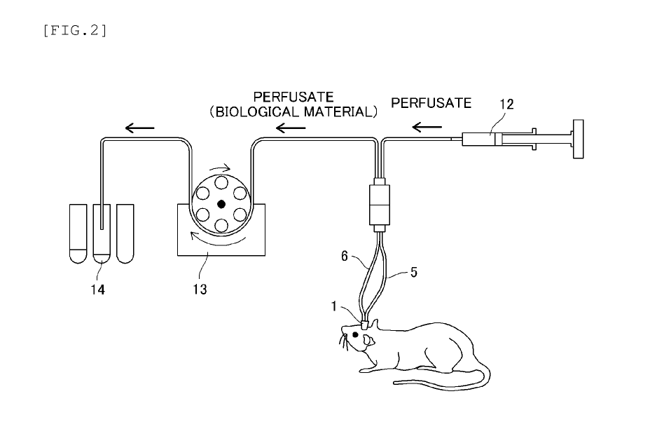

FIG. 2 is a diagram illustrating the dialysis probe 1 according to the first embodiment in use. As shown in the figure, the dialysis probe 1 has its tip (a working dialysis portion of the dialysis membrane 2 that protrudes from the support tube 3) embedded in a brain tissue of a rat. Also, the inlet conduit 5 extending from the dialysis probe 1 is connected to a syringe pump 12, and the outlet conduit 6 extends through a peristaltic pump 13 and is connected to a collector 14.

In this collection system, a perfusate fed from the syringe pump 12 flows at a predetermined inflow rate through the inlet conduit 5 into the space 10 of the dialysis probe 1 to reach the proximity of the tip of the dialysis membrane 2. Then, the inflow perfusate takes in biological materials included in the brain tissue and rises higher within the space 10 to be sucked by the peristaltic pump 13 and thereby to flow out of the space 10 via the outlet conduit 6 at a predetermined outflow rate. The outflow perfusate (containing biological materials) is ultimately collected by the collector 14 and analyzed by an analyzer.

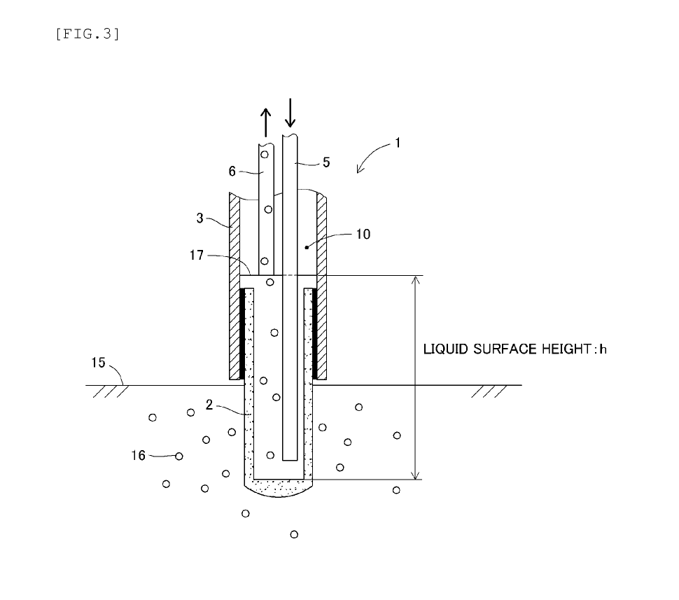

FIG. 3 is a diagram describing the operating principle of the dialysis probe 1 according to the first embodiment. As is apparent from the figure, in the case of the dialysis probe 1 according to the present embodiment, when the perfusate flows into the space 10 via the inlet conduit 5, the liquid surface 17 of the perfusate reaches the tip of the outlet conduit 6. Then, as the perfusate further flows in, the perfusate, which contains biological materials 16 that are derived from a body tissue 15 and transmitted through the dialysis membrane 2, is sucked by the peristaltic pump 13 and flows out via the outlet conduit 6.

For the dialysis probe 1 according to the present embodiment, the outflow and inflow rates of the perfusate do not have to be equal but the pumps need to be set such that the outflow rate is greater than or equal to the inflow rate. Such settings make it possible to maintain the height h of the liquid surface 17. Note that when the perfusate outflow rate is greater than the inflow rate, the perfusate is sucked into the tip of the outlet conduit 6, along with air taken into the space 10 from the air-exposure through-hole 7. Accordingly, in such a case also, the internal pressure of the dialysis membrane 2 can be maintained at a hydraulic pressure level proportional to the liquid surface height h.

To sum up the foregoing, the dialysis probe 1 according to the present embodiment maintains the internal pressure of the dialysis membrane 2 at a hydraulic pressure level proportional to the liquid surface height h, making it possible to stably perform an analysis with high accuracy without causing the amount (concentration) of biological material collected to temporarily increase/decrease due to the internal pressure of the dialysis membrane 2 being increased/decreased and without the perfusate leaking into a body tissue.

Also, the liquid surface height h can be set as low as about 1 mm plus the length of the dialysis membrane 2 by changing the protruding length of the outlet conduit 6. Therefore, the dialysis probe 1 according to the present embodiment makes it possible to analyze biological materials of relatively high molecular weight using the dialysis membrane 2 to such a cutoff value as not to cause a perfusate to leak under hydraulic pressure corresponding to h.

Described next are the results of in vitro analyzing the amount (concentration) of beta amyloid using the dialysis probe 1 according to the present embodiment. Principal experimental conditions were as follows:
the inflow rate of an artificial tissue fluid (perfusate): 1.0 μL/min.;
the outflow rate of the artificial tissue fluid (perfusate): 1.0 μL/min. or more;
dialysis membrane: 330 μm in inner diameter, 430 μm in outer diameter, 0.3 μm in average pore diameter;
dialysis membrane length (protruding length from a support tube): 4 mm; and
external liquid: beta amyloid standard solution (concentration=91.1 nM).

The following table shows analysis results for the amount (concentration) of beta amyloid included in a perfusate collected every hour under the conditions specified above.

TABLE 1

| | Amount of Beta | |
|---|---|---|
| Time | Amyloid (nM) | Collection Rate (%) |
| After 1 Hour | 3.13 | 3.44 |
| After 2 Hours | 3.52 | 3.87 |
| After 3 Hours | 3.76 | 4.12 |
| After 4 Hours | 4.75 | 5.21 |
| After 5 Hours | 4.30 | 4.72 |
| External Liquid | 91.10 | 100.00 |

As is apparent from the above table, the dialysis probe 1 according to the present embodiment allowed a 5-hour stable analysis without any significant variations in the amount (concentration) of beta amyloid collected. Also, the collection rate was stable between about 3 to about 5% of the amount (concentration) of beta amyloid included in the external liquid being considered 100%.

### Second Embodiment

### Type for Organ

A dialysis probe 20 according to a second embodiment of the present invention is mainly used for collecting and analyzing biological materials in organs and subcutaneous tissues, and includes a tubular dialysis membrane 21 open at opposite ends, an inlet conduit 22 having its tip coupled to one end of the dialysis membrane 21, an extension tube 23 concentrically coupled to the other end of the dialysis membrane 21 and extending in the axial direction of the dialysis membrane 21, and an outlet conduit 24 extending inside the extension tube 23 from a space 29 within the dialysis membrane 2 out beyond the extension tube 23, as shown in FIG. 4(A). As shown in the figure, the outer diameter of the outlet conduit 24 is smaller than the inner diameters of the dialysis membrane 21 and the extension tube 23. Also, the inlet conduit 22, the extension tube 23, and the outlet conduit 24 are made of, for example, a proper resin material which has flexibility, such as polyethylene.

The dialysis membrane 21 is a hollow fiber dialysis membrane with an outer diameter of hundreds of μm and a thickness of tens of μm. In the present embodiment, the dialysis membrane 21 is made by cutting a tubular dialysis membrane for use in artificial dialysis to a predetermined length.

The outer diameter of the inlet conduit 22 is slightly smaller than the inner diameter of the dialysis membrane 21, and the inner circumferential surface of the dialysis membrane 21 and the outer circumferential surface of the inlet conduit 22 are joined by an adhesive layer 26 provided therebetween. The adhesive layer 26 is made of, for example, an epoxy-based adhesive. There are a variety of conceivable variants on the coupling form of the dialysis membrane 21 and the inlet conduit 22; for example, the inner diameter of the inlet conduit 22 may be slightly larger than the outer diameter of the dialysis membrane 21 so that the inner circumferential surface of the inlet conduit 22 and the outer circumferential surface of the dialysis membrane 21 can be joined by the adhesive layer 26.

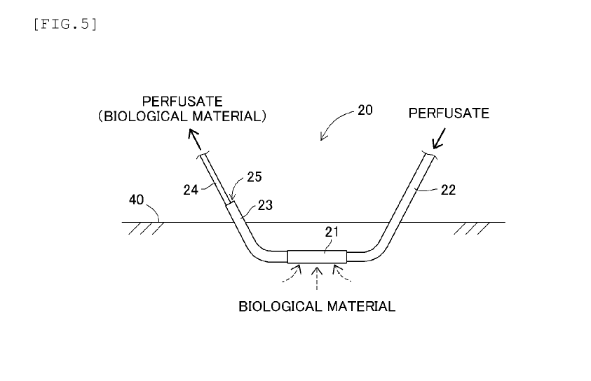

While the outlet conduit 24 has a flat tip, it can be shaped like a needle tip. As a result, it becomes easy to allow the dialysis probe 20 to pierce through a body tissue 40, such as an organ, from the tip of the outlet conduit 24 and secure the dialysis membrane 21 in proximity to the surface of the body tissue 40 (see FIG. 5).

The outer diameter of the extension tube 23 is slightly smaller than the inner diameter of the dialysis membrane 21, and the outer circumferential surface of the extension tube 23 and the inner circumferential surface of the dialysis membrane 21 are joined by an adhesive layer 27 provided therebetween. The adhesive layer 27 is made of, for example, an epoxy-based adhesive.

The outer diameter of the outlet conduit 24 is smaller than the inner diameter of the extension tube 23, and a portion of the inner circumferential surface of the extension tube 23 and a portion of the outer circumferential surface of the outlet conduit 24 are partially joined by an adhesive layer 28 provided therebetween. As a result, an air-exposure gap 25 can be formed between the outlet conduit 24 and the extension tube 23 to maintain the space 29 within the dialysis membrane 21 at atmospheric pressure. The air-exposure gap 25 corresponds to the air-exposure through-hole 7 of the dialysis probe 1 according to the first embodiment.

It can be said that the extension tube 23 and the outlet conduit 24 are inserted in the dialysis membrane 21, and it is preferable that the inserted length of the outlet conduit 24 be shorter than that of the extension tube 23, as shown in FIG. 4(A). In the case where the inserted length of the outlet conduit 24 is longer than that of the extension tube 23, if the perfusate inflow rate is greater than the outflow rate, air taken in through the air-exposure gap 25 might be introduced into the space 29 within the dialysis membrane 21, affecting biological material collection.

There are a variety of conceivable variants on the form of joining the dialysis membrane 21, the extension tube 23 and the outlet conduit 24. For example, as shown in FIG. 4(B), the inner diameter of the extension tube 23 may be set to be slightly larger than the outer diameter of the dialysis membrane 21, so that the inner circumferential surface of the extension tube 23 and the outer circumferential surface of the dialysis membrane 21 can be joined by the adhesive layer 27, and the outer diameter of the outlet conduit 24 may be set to be smaller than the inner diameter of the dialysis membrane 21, so that the outlet conduit 24 and the dialysis membrane 21 can be partially joined by the adhesive layer 28 provided between a portion of the inner circumferential surface of the dialysis membrane 21 and a portion of the outer circumferential surface of the outlet conduit 24. In this case also, the air-exposure gap 25 is formed between the outlet conduit 24 and the extension tube 23 and also between the outlet conduit 24 and the dialysis membrane 21, making it possible to maintain the space 29 at atmospheric pressure.

In addition, as shown in FIG. 4(C), the dialysis membrane 21 and the extension tube 23 may be approximately equal in inner diameter, and the inner circumferential surface of the dialysis membrane 21 and the inner circumferential surface of the extension tube 23 may be joined to the outer circumferential surface of a coupling tube 30 by the adhesive layer 27, so that the dialysis membrane 21 and the extension tube 23 are coupled together via the coupling tube 30. In this case, the outer circumferential surface of the outlet conduit 24 and the inner circumferential surface of the coupling tube 30 are partially joined by the adhesive layer 28, thereby forming the air-exposure gap 25 for exposing the space 29 to air.

In another variant, the dialysis probe 20 shown in FIG. 4(D) includes an outlet conduit 24 and an air-exposure tube 31, which extend from the space 29 within the dialysis membrane 21 in an opposite direction to the inlet conduit 22. The air-exposure tube 31 is intended to expose the space 29 to air and is made of, for example, a proper resin material which has flexibility, such as polyethylene.

The outlet conduit 24 and the air-exposure tube 31 are joined to the inner circumferential surface of the dialysis membrane 21 by an adhesive layer 32, respectively, and the outlet conduit 24 and the air-exposure tube 31 are also joined together by the adhesive layer 32. It can be said that the outlet conduit 24 and the air-exposure tube 31 are inserted in the space 29, penetrating through the adhesive layer 32 for sealing an end of the dialysis membrane 21. As shown in the figure, the inserted length of the air-exposure tube 31 is longer than that of the outlet conduit 24. In addition, the adhesive layer 32 is made of, for example, an epoxy-based adhesive.

In still another variant, the outlet conduit 24 as shown in FIGS. 4(B) and 4(C) can be joined to the inner circumferential surface of the extension tube 23 by the adhesive layer 27. In this case also, by setting the outer diameter of the outlet conduit 24 to be smaller than the inner diameter of the extension tube 23, it becomes possible to form the air-exposure gap 25.

FIG. 5 is a diagram illustrating the dialysis probe 20 according to the second embodiment in use. As shown in the figure, the dialysis membrane 21 is embedded and secured in proximity to the surface of the body tissue 40, and the extension tube 23, the outlet conduit 24, and the inlet conduit 22 extend from either end of the dialysis membrane 21 embedded in the body tissue 40 to the outside of the body tissue 40. In the case of the dialysis probe 20 shown in FIG. 4(D), the inlet conduit 22, the outlet conduit 24, and the air-exposure tube 31 extend to the outside of the body tissue 40.

As in the case of the dialysis probe 1 according to the first embodiment, the dialysis probe 20 according to the present embodiment makes it possible to maintain the space 29 within the dialysis membrane 21 at atmospheric pressure by setting the rate of outflow by the peristaltic pump to be greater than or equal to the rate of perfusate inflow by the syringe pump. Specifically, even if the inflow and outflow rates of the perfusate are not completely equal, it is possible to stably perform an analysis with high accuracy without causing the amount (concentration) of biological material collected to temporarily increase/decrease due to the internal pressure of the dialysis membrane 21 being increased/decreased and without the perfusate leaking into a body tissue.

1: A dialysis probe comprising:
a tubular dialysis membrane sealed at its tip;
a support tube coupled at a tip to a rear end of the dialysis membrane;
a cap portion for securing a rear end of the support tube;
an inlet conduit for guiding a perfusate into a space enclosed by the dialysis membrane, the support tube, and the cap portion, the inlet conduit extending through the cap portion toward the tip of the dialysis membrane within the space;
an outlet conduit for guiding the perfusate in the space outside the space, the outlet conduit extending through the cap portion toward the tip of the dialysis membrane within the space; and
at least one air-exposure through-hole provided in the cap portion for maintaining the space at atmospheric pressure, wherein,
the inlet conduit has a longer protruding length from the rear end of the support tube when compared to the outlet conduit, and an outflow rate of the perfusate exiting the space through the outlet conduit is set to be greater than or equal to an inflow rate of the perfusate entering the space through the inlet conduit.

2: The dialysis probe according to claim 1, wherein the dialysis membrane and the support tube are joined by an adhesive layer provided between an outer circumferential surface of the dialysis membrane and an inner circumferential surface of the support tube.

3: A dialysis probe comprising:
a tubular dialysis membrane open at opposite ends;
an inlet conduit coupled at a tip to one end of the dialysis membrane so as to guide a perfusate into a space within the dialysis membrane; and
an outlet conduit for guiding the perfusate in the space within the dialysis membrane outside the space, the outlet conduit extending from the space in an opposite direction to the inlet conduit, wherein,
the space is exposed to air, and an outflow rate of the perfusate exiting the space through the outlet conduit is set to be greater than or equal to an inflow rate of the perfusate entering the space through the inlet conduit.

4: The dialysis probe according to claim 3, further comprising an air-exposure tube extending from the space in the same direction as the outlet conduit.

5: The dialysis probe according to claim 3, further comprising an extension tube concentrically coupled to the other end of the dialysis membrane and extending in an axial direction of the dialysis membrane, wherein,
the outlet conduit extends inside the extension tube.

6: The dialysis probe according to claim 3, wherein the inlet conduit and the dialysis membrane are joined by an adhesive layer provided between an outer circumferential surface of the inlet conduit and an inner circumferential surface of the dialysis membrane.

7: The dialysis probe according to claim 5, wherein the extension tube and the dialysis membrane are joined by an adhesive layer provided between an outer circumferential surface of the extension tube and an inner circumferential surface of the dialysis membrane, and the extension tube and the outlet conduit are partially joined by an adhesive layer provided between a portion of an inner circumferential surface of the extension tube and a portion of an outer circumferential surface of the outlet conduit.
